(19) United States
(12) Patent Application Publication
(10) Pub. No.: US 2025/0375565 A1
(43) Pub. Date: Dec. 11, 2025

(54) METHOD OF REMOVING FLUID DURING A MEDICAL PROCEDURE AND FLUID REMOVAL APPARATUS

(71) Applicant: The Regents of the University of Michigan, Ann Arbor, MI (US)

(72) Inventors: Neal Podhuturi, Rochester, MI (US); Hannah Pham, Ann Arbor, MI (US); Eric S. Bui, Ann Arbor, MI (US); Demetri A. Monovoukas, Boston, MA (US); Rija R. Awan, Troy, MI (US); Syed Sikander Raza, Ypsilanti, MI (US); Yamaan S. Saadeh, Ann Arbor, MI (US); Yimeng Zhao, Ann Arbor, MI (US); Michael Albdewi, Brooklyn, NY (US)

(21) Appl. No.: 19/230,244

(22) Filed: Jun. 6, 2025

Related U.S. Application Data
(60) Provisional application No. 63/657,074, filed on Jun. 6, 2024.

(51) Int. Cl. A61M 1/00; A61M 25/10; A61M 39/28

(52) U.S. Cl. CPC A61M 1/915; A61M 1/96; A61M 39/28; A61M 25/10; A61M 2202/0413; A61M 2210/0693

(57) ABSTRACT

A medical fluid removal apparatus and method uses negative pressure intraoperatively for the induction of hemostasis. The medical fluid removal apparatus includes a tissue interface membrane, a suction compliant body coupled to the tissue interface membrane, and an adapter configured for attachment to a negative pressure source. The suction compliant body is coupled between the tissue interface membrane and the adapter, and the tissue interface membrane is more flexible than the suction compliant body. The method of removing fluid during a medical procedure can include the steps of placing a tissue interface membrane of a medical fluid removal apparatus against a tissue of a patient, and suctioning the fluid beneath the tissue interface membrane or at least partially around the tissue interface membrane and through a suction compliant body of the medical fluid removal apparatus.

### TECHNICAL FIELD

The present disclosure relates generally to medical fluid removal systems, and more particularly, to vacuum hemostatic methods and devices for use in surgical procedures.

### BACKGROUND

Bleeding is a common intraoperative complication during open surgery. Intraoperative bleeding can obstruct the surgical field, complicating the surgeon's view and creating a more challenging and time-consuming procedure. This can potentially lead to increased mortality risk for patients and higher cost of care. Existing manual hemostasis methods such as cautery and cannula suction have varying success rates based on bleed severity and location, while coagulative agents can be costly.

Surgeons were surveyed to assess the challenges encountered from intraoperative surgical bleeding (thoracic, transplant, neurosurgery, acute care, and vascular surgeons). All of the surveyed surgeons reported inadequate surgical field visualization during bleeding as a major challenge. Localization of bleeding was also reported as a challenge of bleed control by most surgeons, and some reported dissatisfaction with the inefficiencies of losing a clinician's helping hand (e.g., to control bleeding with a suction cannula) during a procedure.

The apparatus and methods described herein are particularly advantageous for use during intracranial tumor resections. Annually, there are more than 15,000 brain tumor resections. Neurosurgical tumor excision involves highly vascular tissue, which presents a high bleed risk. Intraoperative bleeding can obstruct the main operative procedure for surgeons, potentially leading to decreased surgical efficacy. Current hemostatic methods are not always satisfactory. For example, a suction cannula requires active manipulation and can cause injury. Cautery requires precise bleed visualization, and is not a possible option for all tissues. Localization is imprecise with lap pads, and coagulative agents may require retrieval and can be costly. Additionally, chemicals may interfere with the surgical process. Options such as clipping and suture are not feasible for all tissues, particularly neural tissue. Accordingly, there is a need for a surgical apparatus that can adapt to the shape of the surgical field, control bleeding without need for precise localization, and remain operative in a hands-free manner.

### SUMMARY

In accordance with one embodiment, there is provided a method of removing fluid during a medical procedure. The method can include the steps of placing a tissue interface membrane of a medical fluid removal apparatus against a tissue of a patient, and suctioning the fluid beneath the tissue interface membrane or at least partially around the tissue interface membrane and through a suction compliant body of the medical fluid removal apparatus.

In various embodiments, the medical procedure is a surgical procedure having one or more incisions. The surgical procedure can be an intracranial tumor resection with multi-contoured brain tissue. A suction pressure during the suctioning step can allow for hands-free localization of the tissue interface membrane. The fluid includes blood in some implementation, and the suctioning step can induce hemostasis. The placing step may be accomplished without direct localization of a bleed origin of the patient. The placing step may also include deploying the medical fluid removal apparatus through an incision site.

In various embodiments, the tissue interface membrane is a perimeter balloon, which may be inflated after deployment. A material of the tissue interface membrane may be more flexible than a material of the suction compliant body, and the suction compliant body can have a non-convex profile that is straight tapered or slopes inward toward an interior suction chamber. The medical fluid removal apparatus can further include an adapter configured for coupling with a negative pressure source, where the suction compliant body is coupled between the tissue interface membrane and the adapter. A pressure valve can be located between the adapter and the negative pressure source, where the pressure valve is manually operable to change an induced negative pressure during the suctioning step. An induced negative pressure amount during the suctioning step may correlate with a spread area of the tissue interface membrane.

In accordance with another embodiment, there is provided a medical fluid removal apparatus. The medical fluid removal apparatus includes a tissue interface membrane, a suction compliant body coupled to the tissue interface membrane, and an adapter configured for attachment to a negative pressure source. The suction compliant body is coupled between the tissue interface membrane and the adapter, and the tissue interface membrane is more flexible than the suction compliant body.

In various embodiments, the tissue interface membrane is a perimeter balloon. The suction compliant body can have a non-convex profile that is straight tapered or slopes inward toward an interior suction chamber. The negative pressure source can be an operating room suction tubing, with a pressure valve configured to adjust an induced negative pressure. The pressure valve can be an adjustable dial or an adjustable clamp plate configured to clamp the operating room suction tubing.

It is contemplated that any number of the individual features of the above-described embodiments and of any other embodiments depicted in the drawings or description below can be combined in any combination to define an invention, except where features are incompatible.

### BRIEF DESCRIPTION OF THE DRAWINGS

Preferred example embodiments will hereinafter be described in conjunction with the appended drawings, wherein like designations denote like elements, and wherein:

FIG. 1 is a schematic view of one embodiment of a medical fluid removal apparatus, including a negative pressure source;

FIG. 2 is an enlarged view of the medical fluid removal apparatus of FIG. 1;

FIG. 3 shows another example embodiment of a medical fluid removal apparatus;

FIG. 4 shows another example embodiment of a medical fluid removal apparatus;

FIG. 5 shows another example embodiment of a medical fluid removal apparatus;

FIG. 6 shows another example embodiment of a medical fluid removal apparatus;

FIG. 7 is a schematic view of yet another embodiment of a medical fluid removal apparatus, including a negative pressure source;

FIG. 8 is a schematic view of yet another embodiment of a medical fluid removal apparatus in a deflated state, including a negative pressure source;

FIG. 9 is a schematic view of the medical fluid removal apparatus of FIG. 9 in an inflated state;

FIG. 10 shows example dimensions for the medical fluid removal apparatus of FIG. 3;

FIG. 11 shows example dimensions for the medical fluid removal apparatus of FIG. 3; and

FIG. 12 shows another example of a pressure valve that can be used with a medical fluid removal apparatus.

### DESCRIPTION OF THE ILLUSTRATED EMBODIMENTS

Described herein is a medical fluid removal apparatus and method of controlling a patient's bleeding during a medical procedure. In an advantageous embodiment, the apparatus uses negative pressure intraoperatively for the induction of hemostasis. Embodiments also improve the ergonomics and efficacy of surgical suction. Apparatus designs provide for adhesion with, and fluid removal from, a multi-contoured tissue surface, allowing for hands-free operation once the apparatus is placed on the tissue to be treated. The apparatus can also better adapt to the shape of the surgical field, controlling bleeding without the need for precise localization.

The apparatus and methods described herein are particularly applicable for intracranial tumor resections, given the high tissue vascularity and small operative field. While it should be understood that the apparatus and methods are applicable to other surgical and medical procedures, the present disclosure was particularly developed to remedy the deficiencies of current methods of bleed control used by neurosurgeons during solid tumor resection, which can be expensive and cumbersome. The present apparatus and methods provide tissue-compatible bleed control during intracranial tumor resection in a cost-effective and ergonomic way.

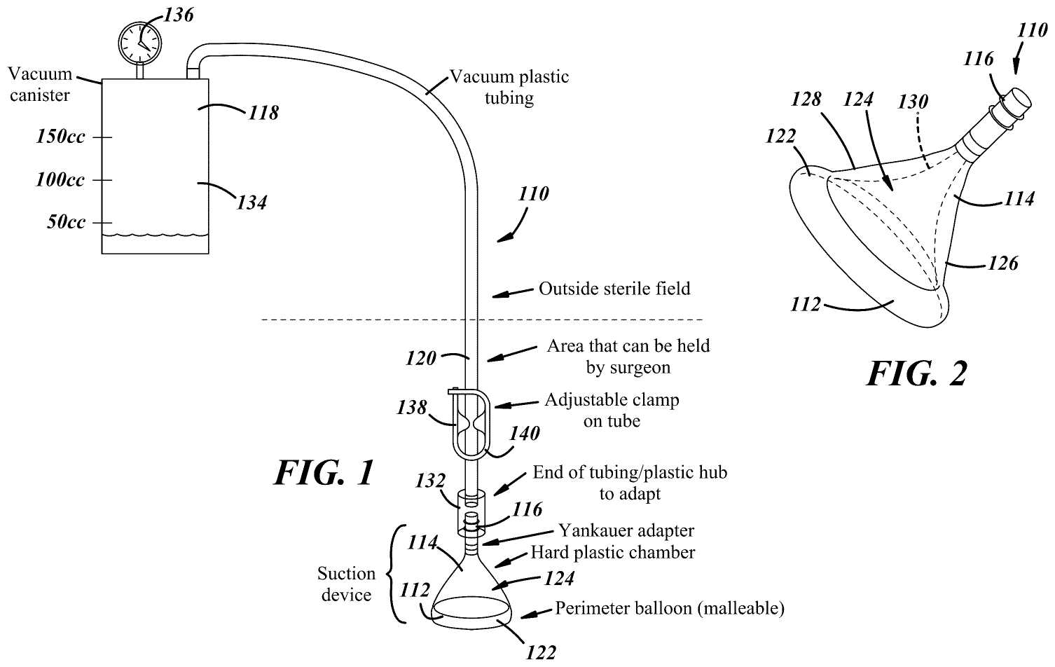

FIGS. 1 and 2 schematically illustrate an example medical fluid removal apparatus 110 that can be used to control bleeding of a patient during a medical procedure, such as an intracranial tumor resection surgical procedure, to cite one example. It should be noted that the apparatus 110 can be used in other applications. For example, the apparatus 110 may be particularly useful in open surgical procedures, in which a surgeon makes an incision to gain direct access to internal organs and tissues (e.g., intra-abdominal surgery). In other embodiments, the apparatus 110 may be used less invasive procedures, such as a laparoscopic procedure to cite one example.

The medical fluid removal apparatus 110 includes a tissue interface membrane 112, a suction compliant body 114, and an adapter 116. The apparatus 110 may also include a negative pressure source 118, or the adapter 116 may be configured to couple with a separate negative pressure source, such as standard operating room (OR) tubing 120 in one example. In some embodiments, the apparatus 110 may include its own dedicated negative pressure source 118, but providing for connection with standard OR tubing 120 may be more operationally feasible.

Unlike a cannula or the like, the tissue interface membrane 112 can be included with the suction compliant body 114 to more adaptably contour to the patient's tissue. In the illustrated embodiment, the tissue interface membrane 112 is a perimeter balloon 122. The tissue interface membrane 112 is preferably manufactured from a more flexible material than the suction compliant body 114, and may take the form of a cushion-type perimeter balloon 122 as illustrated, or more of a flap-type seal, foam perimeter layer, or another operable, conformable structure, to cite a few examples. Having the tissue interface membrane 112 as a more flexible or malleable material can help achieve better conformability, while maintaining enough rigidity at the suction compliant body 114 such that the apparatus 110 can suitably control bleeding at the patient's tissue.

In one implementation, to accomplish the variance in flexibility between the tissue interface membrane 112 and the suction compliant body 114, the tissue interface membrane is made from a flexible material such as silicone or rubber, and the suction compliant body is made from a more rigid material, such as polysulfone plastic, polyvinyl chloride (PVC), or neoprene, to cite a few examples. In one advantageous embodiment, the tissue interface membrane 112 is made from natural rubber, the suction compliant body 114 is made from polysulfone plastic, and the adapter 116 is made from PVC. Varying the rigidity between the adapter 116 (most rigid) to the suction complaint body 114 (intermediate) to the tissue interface membrane 112 (most flexible) allows for conformational adaptation to the target tissue while facilitating a secure connection with tubing 120.

In another example embodiment, the thickness of each of the tissue interface membrane 112 and the suction compliant body 114 can be adjusted to vary the flexibility. For example, the tissue interface membrane 112 can be made from a thinner material that is fluid backed, like the perimeter balloon 122. In another implementation, two different materials may be used to make the tissue interface membrane 112 and the suction compliant body 114, with flexibility being imparted by using a material with a lower elastic modulus for the tissue interface membrane 112. In some embodiments, transparent or semi-transparent materials for the tissue interface membrane 112 and/or the suction compliant body 114 may help with visualization.

Unlike a cannula, the tissue interface membrane 112 is configured to minimize potential tissue damage. However, given its larger surface area (e.g., about 2.5 cm radial extension for the perimeter balloon 122, surrounding an interior suction chamber 124 under the suction compliant body 114 that is about 7-16 mm² in area adjacent an interior edge of the tissue interface membrane 112), fluid dynamic control can potentially be more challenging. The size and shape of the medical fluid removal apparatus 110 impacts the fluid dynamic properties, and in at least some embodiments, the amount of negative pressure used is correlated with the size of the apparatus. For example, a spread size of the tissue interface membrane 112 may be used to determine an adequate negative pressure to be applied. A smaller spread area or radius for the tissue interface membrane 112 would need less pressure than with a larger spread area or radius. Here, given the 2.5 cm radial extension for the perimeter balloon, a spread area would be about 50-70 mm² and the induced negative pressure may be about 7-8 N to allow for hands-free operation. In fluid collection and adhesion force tests, adequate fluid collection and adhesion occurred at about 7.8 N given this apparatus 110 structure. Additionally, given the larger spread area of the perimeter balloon 122 as opposed to a cannula or the like, the apparatus 110 can be placed in the surgical filed without direct localization of a bleed origin of the patient. This configuration can also help improve the induction of hemostasis as suction is applied.

When removing blood in particular, clotting can be a challenge, and areas of stagnation can be problematic. Accordingly, the size or spread area of the apparatus 110 might change depending on the desired surgical procedure and target tissue. Other dimensions can be varied depending on the surgical procedure. For example, a height of the perimeter balloon 122 may be changed, or the fluid within the balloon may be altered. In the illustrated embodiment, air is used as the fluid in the perimeter balloon 122, but other fluids may be used, such as a more gelatinous fluid to cite one example. The amount of fluid in the perimeter balloon 122 may also be varied to alter the flexibility of the tissue interface membrane 112. The tissue interface membrane 112 in FIGS. 1 and 2 is particularly configured for multi-contoured attachment to brain tissue, but the dimensions and configuration may be adjusted with other target tissues and/or surgical procedures. Additionally, it should be noted that “removal” as used herein means removal of fluid from the patient's wounded area or movement of fluid around the patient's tissue (to help promote coagulation, for example, with blood), not necessarily that the fluid needs to be removed and entirely sucked away from the tissue and/or through the tubing 120.

In the illustrated embodiment, the suction compliant body 114 is directly coupled to the tissue interface membrane 112 and serves as an intermediate portion between the tissue interface membrane 112 and the adapter 116. As shown more particularly in FIG. 2, the suction compliant body 114 helps define the interior suction chamber 124. The suction compliant body 114 in this embodiment has a non-convex profile 126 that is straight tapered from the largest radial extent at the tissue interface membrane 112 to the smallest radial extent at the adapter 116. When suction or negative pressure is applied, the conical wall 128 of the suction compliant body 114 may bow or slope inward as shown with dotted lines at 130. In some embodiments, the non-convex profile 126 may include the inward slope 130 at rest instead of having a straight taper. The amount of movement of the suction compliant body 114 during operation will depend on the amount of induced negative pressure, the dimensions and shape of the apparatus 110, and/or the material used for the suction compliant body, to cite a few factors. Some movement, particularly from a straight taper to an inward slope 130 or from an initial inward slope to a greater inward slope (i.e., a radial contraction), is desirable, as it helps create a more positive attachment at the tissue interface membrane 112, which can then provide for hands-free operation if desired. Additionally, this non-convex profile 126 shape can help improve fluid flow dynamics to help better induce hemostasis. However, other shapes are certainly possible for the suction compliant body 114, such as a pyramid shape to cite one example.

The adapter 116 is used to help couple the tissue interface membrane 112 and suction compliant body 114 to the negative pressure source 118. The adapter 116 can be used to modulate pressure and help disperse suction over a larger spread area. In an advantageous embodiment, the adapter 116 is a Yankauer adapter, which can be particularly useful with blood, a coagulative fluid. Other adapter structures can be used, preferably catheter-style structures are configured to aspirate potentially thicker fluids. The adapter 116 is typically less flexible than both the tissue interface membrane 112 and the suction compliant body 114, which can help facilitate attachment to the negative pressure source 118 via the tubing 120. Other connection features, such as a hub 132 or adapter sleeve, threads, clips, or other fasteners, etc. may be included as the adapter 116.

Negative pressure source 118 provides a vacuum to move fluid from the patient's tissue. The apparatus 110 is advantageously a vacuum hemostasis surgical device that is configured to suction fluid that is located around and/or beneath the tissue interface membrane 112. In some implementations, the apparatus 110 comes with its own negative pressure source 118, and in other implementations, the apparatus 110 comes with the tissue interface membrane 112, suction compliant body 114, and adapter 116 which then attaches to tubing 120 for a separate negative pressure source 118. This arrangement may be beneficial, as it allows for the apparatus 110 to be largely disposable and/or sterilizable, then hook up to standard OR tubing 120. As shown in FIG. 1, this arrangement includes a vacuum canister 134 and at least a portion of the tubing 120 to be in a sterile environment, and the tubing 120 with the apparatus 110 can be held in the operating room. The negative pressure source 118 may also include or be coupled to other operable features, such as a pressure gauge 136 and/or a pressure valve 138, to cite a few examples.

The pressure gauge 136 can provide a visual, auditory, and/or haptic output to help a clinician ascertain the induced negative pressure from the negative pressure source 118, and the pressure valve 138 may be included to alter the amount of applied negative pressure. The hub 132 may help hold the pressure valve 138 in place during shipment of the apparatus 110, and then the valve can interact with the tubing 120 to increase or decrease the amount of applied negative pressure. In the illustrated embodiment, the pressure valve 138 is an adjustable clamp plate 140 located between the adapter 116 and the negative pressure source 118 that can be manually adjusted to clamp or constrict the tubing 120, thereby altering the amount of applied negative pressure. The adjustable clamp plate 140 may be advantageous as it can potentially remove suction pressure at a rate of about 100 ccs per five seconds. Other gauges 136, valves 138, sensors, etc. may be included, such as a digital flow meter in one example embodiment.

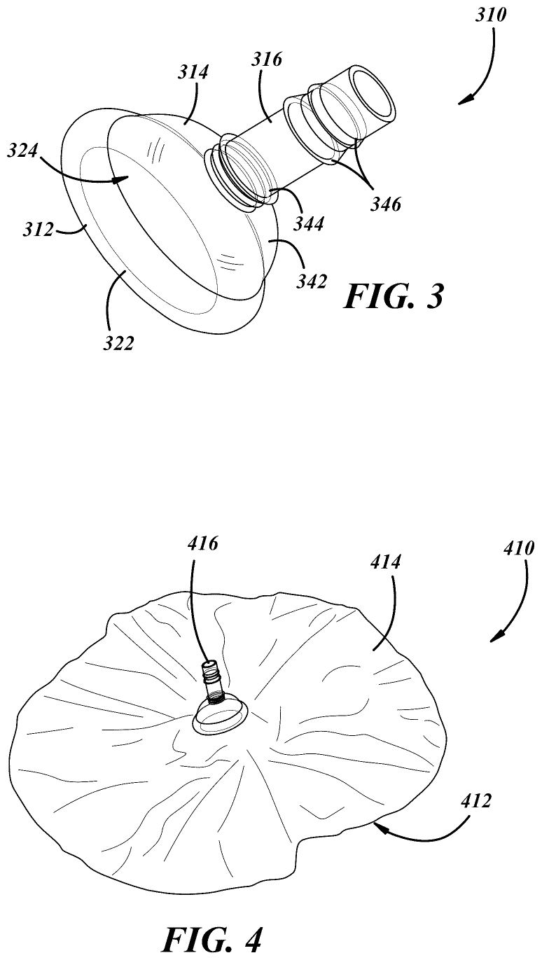

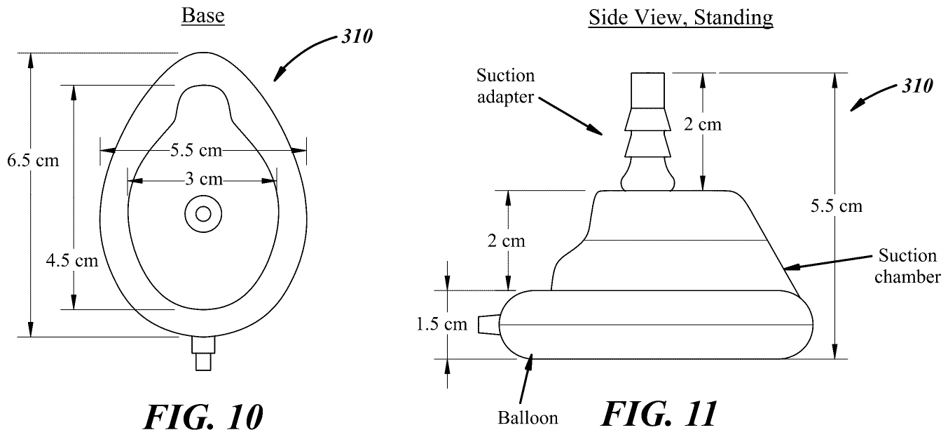

FIG. 3 shows another embodiment of a medical fluid removal apparatus 310 (wherein like reference numerals denote like features). In this embodiment, the suction compliant body 314 has a convex profile 342 as opposed to the non-convex profile 126 of FIGS. 1 and 2. This arrangement increases the size of the interior suction chamber 124. This embodiment 310 also includes a threaded engagement 344 between the suction compliant body 314 and the adapter 316, but it is feasible in other implementations to have the adapter integrally extend from the suction compliant body. The adapter 316 in this implementation includes a plurality of radial ribs 346, which may be used to help retain other subcomponents, such as the hub, valve, or tubing illustrated in conjunction with the FIG. 1 embodiment. The embodiment 310 also includes a perimeter balloon 322 for the tissue interface membrane 312, which was shown in testing to be adequate for fluid collection and adhesion force (e.g., about 783 g). Example dimensions for the embodiment illustrated in FIG. 3 are shown in FIGS. 10 and 11.

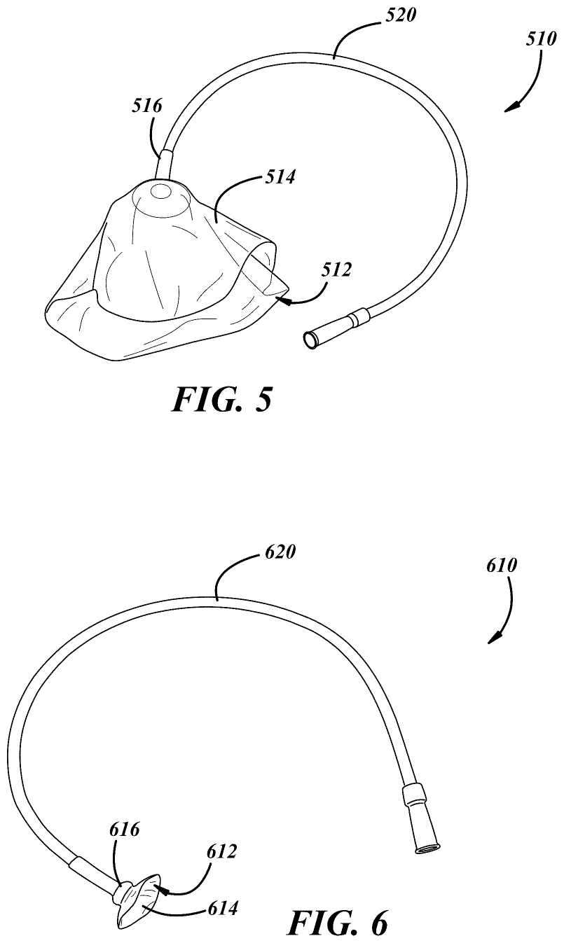

FIG. 4 shows an embodiment of a medical fluid removal apparatus 410, and FIG. 5 shows a similar embodiment of a medical fluid removal apparatus 510. With these embodiments, a biocompatible coating or adhesive layer or the like may be included as the tissue interface membrane 412, 512, particularly given the flexible skirt-like structure of the suction complaint body 414, 514. The apparatus 410 has a 0° skirt for the suction compliant body 414, and the apparatus 510 has a 90° skirt for the suction compliant body 514. These iterations were less successful in adhesion force testing, and thus adaptations can be made to enhance their feasibility in various surgical procedures.

FIG. 6 shows another embodiment of a medical fluid removal apparatus 610. This embodiment has more of a suction-cup like structure for the suction compliant body 614, without a perimeter balloon as the tissue interface membrane 612. Instead, a biocompatible coating, thin film, thin foam, or other operable layer can be used as the tissue interface membrane 612. Different sizes of the apparatus 610 were tested (e.g., 20 mm, 30 mm, and 40 mm), and all were sufficient for fluid collection. Adjustments could be made to improve adhesion force, although the adhesion force was improved compared with the embodiments shown in FIGS. 4 and 5.

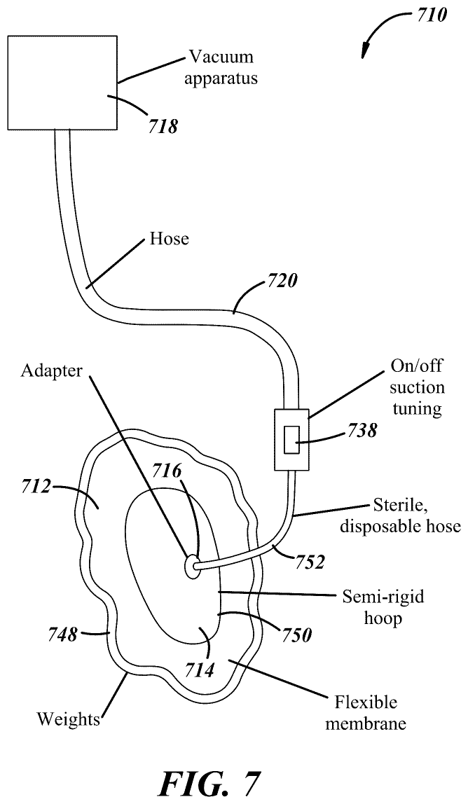

FIG. 7 shows yet another embodiment of a medical fluid removal apparatus 710. In this implementation, the tissue interface membrane 712 has more of a flap-style structure with a weighted outer perimeter 748. The weighted outer perimeter 748 may be an edge of a different material, or have a plurality of discrete weighted portions or weights around the outer edge, to cite a few examples. This embodiment also includes a semi-rigid hoop 750 that delineates the membrane 712 from the suction compliant body 714. This can help define the interior suction chamber 724. The apparatus 710 further includes a sterile, disposable hose 752 which is coupled between the adapter 716 and pressure valve 738.

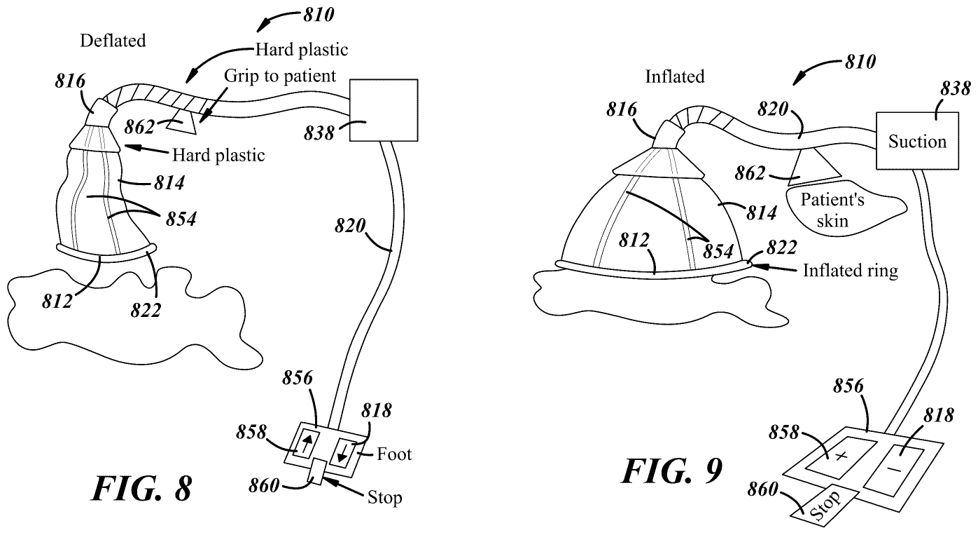

FIGS. 8 and 9 illustrate yet another embodiment of a medical fluid removal apparatus 810. This embodiment may be more useful in a laparoscopic procedure, for example, potentially being deployed via a catheter into a smaller incision if there is undesirable bleeding during a surgical procedure. The size of the apparatus 810 would need to be considered and adjusted accordingly for adaptation to such an application. FIG. 8 shows the apparatus 810 in a deflated state, and FIG. 9 shows the apparatus inflated, with one or more fluid channels 854 operably connecting the perimeter balloon 822 to a positive pressure source. This allows for the apparatus 810 to be inflated during the medical procedure, as the fluid channels 854 extend between the tissue interface membrane 812 and the adapter 816 along the suction compliant body 814. FIGS. 8 and 9 also schematically represent deployment of the apparatus 810 on a multi-contoured patient tissue, such as brain tissue during tumor resection.

In the implementation shown in FIGS. 8 and 9, the negative pressure source 818 may be part of an overall pressure source 856 that also includes a positive pressure source 858 for inflation of the apparatus 810. The negative pressure source 818 and positive pressure source 858 may be independently operable with a foot pedal or the like as illustrated, with an integrated stop or release 860 that may be configured to stop the induction of positive pressure inflating the balloon 822 and stop the application of negative pressure to release the vacuum in the interior suction chamber 824. This apparatus 810 also includes a separate grip 860 that can be used along the tubing 820 to mount the apparatus to the patient's skin, remote from the incision location. This can further help with improved localization and retention of the apparatus 810 while suctioning.

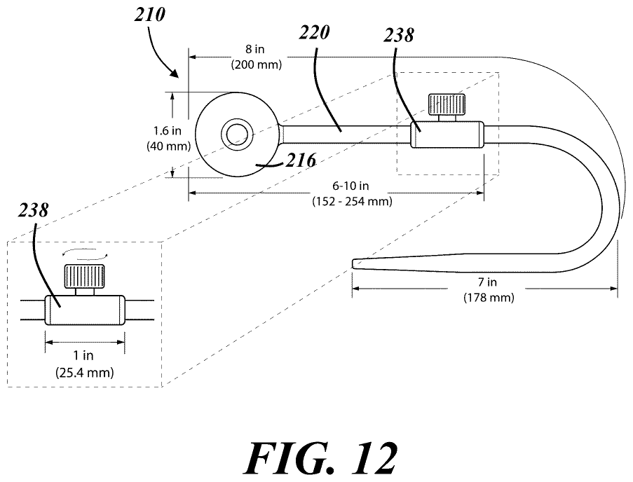

FIG. 12 shows another embodiment of the medical fluid removal apparatus 210. In this implementation, the pressure valve 238 has an adjustable dial configuration that can help with pressure tubing and pressure control within the tubing 220. The pressure valve 238 is located along the tubing 220 between the adapter 216 and the tubing end, which may be coupled to a vacuum source. This pressure valve 238 may be used with the other apparatus embodiments pictured herein, or may have a structure that is not particularly depicted within the figures.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A method of removing fluid of a patient during a medical procedure, comprising the steps of:
placing a tissue interface membrane of a medical fluid removal apparatus against a tissue of the patient; and
suctioning the fluid beneath the tissue interface membrane or at least partially around the tissue interface membrane and through a suction compliant body of the medical fluid removal apparatus.

2. The method of claim 1, wherein the medical procedure is a surgical procedure having one or more incisions.

3. The method of claim 2, wherein the surgical procedure is an intracranial tumor resection and the tissue is multi-contoured brain tissue.

4. The method of claim 1, wherein a suction pressure during the suctioning step allows for hands-free localization of the tissue interface membrane.

5. The method of claim 1, wherein the tissue interface membrane is a perimeter balloon.

6. The method of claim 1, wherein a material of the tissue interface membrane is more flexible than a material of the suction compliant body.

7. The medical fluid removal apparatus of claim 1, wherein the suction compliant body has a non-convex profile that is straight tapered or slopes inward toward an interior suction chamber.

8. The method of claim 1, wherein the medical fluid removal apparatus includes an adapter configured for coupling with a negative pressure source, wherein the suction compliant body is coupled between the tissue interface membrane and the adapter.

9. The method of claim 8, further comprising a pressure valve located between the adapter and the negative pressure source, wherein the pressure valve is manually operable to change an induced negative pressure during the suctioning step.

10. The method of claim 1, wherein an induced negative pressure amount during the suctioning step correlates with a spread area of the tissue interface membrane.

11. The method of claim 1, wherein the fluid includes blood.

12. The method of claim 11, wherein the suctioning step induces hemostasis.

13. The method of claim 1, wherein the placing step is accomplished without direct localization of a bleed origin of the patient.

14. The method of claim 1, wherein the placing step includes deploying the medical fluid removal apparatus through an incision site.

15. The method of claim 14, wherein the tissue interface membrane is a perimeter balloon, further comprising the step of inflating the perimeter balloon after deploying.

16. A medical fluid removal apparatus, comprising:
a tissue interface membrane;
a suction compliant body coupled to the tissue interface membrane; and
an adapter configured for attachment to a negative pressure source, wherein the suction compliant body is coupled between the tissue interface membrane and the adapter, and wherein the tissue interface membrane is more flexible than the suction compliant body.

17. The medical fluid removal apparatus of claim 16, wherein the tissue interface membrane is a perimeter balloon.

18. The medical fluid removal apparatus of claim 16, wherein the suction compliant body has a non-convex profile that is straight tapered or slopes inward toward an interior suction chamber.

19. The medical fluid removal apparatus of claim 16, wherein the negative pressure source is an operating room suction tubing.

20. The medical fluid removal apparatus of claim 19, comprising a pressure valve configured to adjust an induced negative pressure.

21. The medical fluid removal apparatus of claim 20, wherein the pressure valve is an adjustable clamp plate configured to clamp the operating room suction tubing.

22. The medical fluid removal apparatus of claim 20, wherein the pressure valve includes an adjustable dial.
